(19) United States
(12) Patent Application Publication
(10) Pub. No.: US 2026/0008743 A1
(43) Pub. Date: Jan. 8, 2026

(54) PROCESS FOR PRODUCING POLYUNSATURATED FATTY ACIDS IN THE FORM OF ESTERS

(71) Applicant: ENI S.P.A., Roma (IT)

(72) Inventors: Alberto Renato DE ANGELIS, Roma (IT); Luca CHIARABAGLIO, Roma (IT); Lorenzo GUAZZELLI, Firenze (IT)

(73) Assignee: ENI S.p.A., Roma (IT)

(21) Appl. No.: 18/993,663

(22) PCT Filed: Jul. 11, 2023
(86) PCT No.: PCT/IB2023/057099

§ 371 Date: Jan. 13, 2025

(30) Foreign Application Priority Data
Jul. 13, 2022 (IT) 102022000014677

(51) Int. Cl. C07C 67/58

(52) U.S. Cl. CPC C07C 67/58

(57) ABSTRACT

The present invention relates to an integrated process for the simultaneous production of polyunsaturated fatty acids, in particular in the form of esters, and of esters of palmitic acid from algae. The compounds of the present invention can be used in the pharmaceutical industry (products for the prevention of cardio-circulatory problems) and in the cosmetic industry (emollients and moisturizers).

In particular, the present invention is directed to a process for obtaining eicosapentaenoic acid (EPA) and docosahexaenoic acid (DHA) esters from algal material, comprising the steps of:
a) treating the algal material with an extraction solvent to give a liquid phase containing said solvent and algal oil and a solid phase comprising cellulose, sugars and proteins;
b) separating the solid phase from the liquid phase of step a);
c) separating the algal oil from the extraction solvent in the liquid phase of step b);
d) subjecting the algal oil of step c) to a low-temperature treatment so as to obtain the precipitation of saturated and monounsaturated fatty acid triglycerides and a liquid phase containing or consisting of polyunsaturated fatty acid triglycerides;
e1) separating, isolating and purifying DHA and EPA triglycerides from the liquid phase of step d), or
e2) transesterification with ethanol of DHA and EPA triglycerides and isolating the respective DHA and EPA ethyl esters;
f) optionally, isolating and purifying the palmitic acid triglyceride from the phase containing the saturated and monounsaturated fatty acid triglycerides of step d);
g) optionally, transesterification of the palmitic acid triglyceride of step f) with a linear or branched C1-C4 alcohol, to give a C1-C4 alkyl ester of the palmitic acid and glycerol;
h) optionally, hydrogenating the glycerol of step g) to give isopropanol.

### TECHNICAL FIELD OF THE INVENTION

The present invention relates to an integrated process for the simultaneous production of polyunsaturated fatty acids, in particular in the form of esters, and of esters of palmitic acid from algae. The compounds of the present invention can be used in the pharmaceutical industry (products for the prevention of cardio-circulatory problems) and in the cosmetic industry (emollients and moisturizers).

### BACKGROUND ART

The ω-3 polyunsaturated fatty acids (PUFAs), mainly consisting of eicosapentaenoic acid (EPA) and docosahexaenoic acid (DHA), are mainly produced by the fishing industry and to a lesser extent by krill fishing. The strong expansion of the use of PUFAs in the prevention of cardiovascular diseases in the face of an almost static situation in the fishing industry, which is not capable of ensuring an adequate increase in the production of fish oil from which to extract the PUFAs themselves, has increased the demand for these pharmaceutical products and has led to the need to find new supply sources. Such sources could be identified in some algae, in which the PUFA content in the algal bio-oil can even reach up to 15-20%.

Furthermore, the cultivation of oily microalgae allows, by means of chlorophyll photosynthesis, converting CO₂ into organic derivatives, such as sugars or fats, thus reducing the overall content of this greenhouse gas. They are plant microorganisms which accumulate energy reserves in the form of vegetable oil from the sun through chlorophyll photosynthesis, with greater energy efficiency than all other oily plants in nature, accumulating up to 50% of the mass thereof in oil.

In fact, these are the microorganisms which, together with animal organisms, have created oil reserves over the millennia. These microalgae can be grown where food plants cannot be grown: in water (sea, freshwater) and less well in arid and rocky soils and even in the desert. Dry soils can also be used as the passive support of tanks for the hydroculture of microalgae, whereby the cultivation thereof is not to the detriment of normal agriculture, as instead occurs with ethanol from wheat, rice, potatoes, etc. The bio-oil obtained from algal material is normally used in the production of bio-diesel.

Furthermore, the market calls for the replacement of the active ingredients of petrochemical origin of the cosmetic industry with analogous products of biological origin. For example, substitutes for long-chain alkanes used as moisturizers and emollients are sought in creams with products with similar features of biological origin. The esters of palmitic acid with certain alcohols, derivable from glycerin, could be a valid substitute for long-chain alkanes, obtained by the oligomerization of olefins.

Obtaining bio-oil from algae by means of a Deep Eutectic Solvent (DES) is known from WO2020/053118 of the present Applicant.

The extraction of PUFAs from algae by means of supercritical CO₂ is also known.

A. Mendes et al. (DHA Concentration and Purification from the Marine Heterotrophic Microalga Crypthecodinium cohnii CCMP316 by Winterization and Urea Complexation, Food Technol. Biotechnol. 45 (1) 38-44 (2007)) describe a process for obtaining DHA which includes the following steps: saponification and methylation of wet biomass, winterization and complexation with urea.

However, no industrial processes are known which allow the simultaneous production of DHA and EPA in purified form from algae.

It is also unknown to obtain palmitic acid esters from algal material useful in the cosmetic industry.

In other words, there are currently no processes which allow simultaneously exploiting both the fraction consisting of the unsaturated fraction of the triglycerides of the fatty acids and the saturated one: being able to enhance both fractions of the algal oil would make the bio-fixation of CO₂ by means of algae more economically sustainable.

Therefore, there is a need to provide a production process of DHA and EPA from algal material which is industrially applicable and economically advantageous and which preferably also allows obtaining palmitic acid esters usable in the cosmetic industry.

### SUMMARY OF THE INVENTION

Therefore, the present invention relates to:
1) a process for obtaining eicosapentaenoic acid (EPA) and docosahexaenoic acid (DHA) esters from algal material, comprising the steps of:
a) treating the algal material with an extraction solvent to give a liquid phase containing said solvent and algal oil and a solid phase comprising cellulose, sugars and proteins;
b) separating the solid phase from the liquid phase of step a);
c) separating the algal oil from the extraction solvent in the liquid phase of step b);
d) subjecting the algal oil of step c) to a low-temperature treatment so as to obtain the precipitation of saturated and monounsaturated fatty acid triglycerides (precipitate) and a liquid phase containing or consisting of polyunsaturated fatty acid triglycerides;
e1) separating, isolating and purifying DHA and EPA triglycerides from the liquid phase of step d), or
e2) transesterification with ethanol of DHA and EPA triglycerides and isolating the respective DHA and EPA ethyl esters;
f) optionally, isolating and purifying the palmitic acid triglyceride from the phase containing the saturated and monounsaturated fatty acid triglycerides of step d);
g) optionally, transesterification of the palmitic acid triglyceride of step f) with a linear or branched C1-C4 alcohol, to give a C1-C4 alkyl ester of the palmitic acid and glycerol;
h) optionally, hydrogenating the glycerol of step g) to give isopropanol.

The invention is further directed to:
2) a process as in point 1), in which the algal material is selected from algal material from microalgae belonging to the genus Chlorella, Tetraselmis, Nannochloropsis and Scenedesmus, the microalgae of the genus Nannochloropsis and Scenedesmus being preferred;
3) a process as in points 1) or 2), in which the extraction solvent is selected from a chloroform/methanol mixture, preferably in a 2:1 ratio, and a DES (Deep Eutectic Solvent), a DES being the preferred extraction solvent;
4) a process as in point 3), in which the extraction solvent is a low melting temperature eutectic (DES type III) formed by an ammonium salt selected from cholinium chloride, cholinium acetate, cholinium nitrate, betainium chloride, ethyl ammoniochloride, tetramethyl ammonium chloride, and a hydrogen-binding donor selected from a carboxylic acid such as acetic acid, formic acid, tartaric acid, oxalic acid, levulinic acid, citric acid or lactic acid, or an amine such as urea, thiourea, 1,3-dimethylurea, 1,1-dimethylurea, or a polyalcohol such as ethylene glycol, propylene glycol or glycerol, and where the ammonium salt and the hydrogen-binding donor are preferably in a molar ratio of 1:1 to 1:3;
5) a process as in point 4), in which the DES consists of a 1:3 ratio mixture of cholinium chloride and lactic acid or cholinium chloride and citric acid;
6) a process as in any one of points 3) to 5), in which step a) is conducted by treatment of the algal material with a weight amount of DES between 1:1 and 5:1, preferably between 1:1 and 2:1, with respect to the algal material, stirring the dispersion for a period between 30 minutes and 5 hours, preferably between 3 and 5 hours, at temperatures between 80° C. and 130° C., preferably between 90° C. and 110° C.;
7) a process as in any one of points 1) to 6), in which, in step b), the separation of the solid phase is conducted by filtration or centrifugation;
8) a process as in any one of points 1) to 7), in which, in step c), the separation of the algal oil from the extraction solvent is conducted by distillation or by treatment with a strongly solvent, polar washing immiscible with the algal oil but miscible with the extraction solvent;
9) a process as in point 8), in which the extraction solvent is a DES and the washing solvent is water and in which the washing solvent is added to the algal oil solution in DES in a weight ratio preferably between 100 and 300% by weight;
10) a process as in point 9), in which step c) comprises a further step c1) of recycling the extraction solvent in step a); 11) a process as in any one of points 1) to 10), in which step d) consists of a “winterization” with low-temperature fractional crystallization of saturated and mono-unsaturated fatty acid triglycerides (solid phase) from the liquid phase containing poly-unsaturated fatty acid triglycerides, in which the algal oil obtained in step c) is treated at a temperature between 0° C. and −30° C., preferably between −2° C. and −25° C., preferably in the presence of an alcohol and/or urea, more preferably an alcohol;
12) a process as in point 11), in which the alcohol is selected from linear or branched C1-C4 alcohols, and is preferably selected from ethanol, n-butanol and isopropanol, and in which the weight ratio of alcohol with respect to the algal oil weight is preferably between 13:1 and 25:1;
13) a process as in any one of points 1) to 12), in which step e1) is conducted by means of preparative HPLC;
14) a process as in point 13), in which step e1) is conducted by means of a reverse phase preparative HPLC system, using as mobile phase an acetonitrile-chloroform mixture with gradient, or a methanol-water mixture with gradient, and/or using a stationary phase consisting of a PMDVB (Poly-Meta-DiVinyl Benzene) porous resin modified with 3,5-dinitrobenzoyl chloride or modified with 2-chloro ethanol, or consisting of Silver(I)mercaptopropyl (AgTCM);
15) a process as in any one of points 1) to 12), in which the alternative step e2) comprises the transformation of the PUFA triglycerides present in the liquid phase, in particular DHA and EPA triglycerides, into the corresponding ethyl esters by transesterification, in which, when step d) is conducted in the presence of ethanol, said conversion is conducted by adding to the liquid phase an acid catalyst, preferably selected from sulfuric acid, hydrochloric acid, an acid resin or a zeolite and heating to a temperature preferably between 70° C. and 90° C. until completion of the reaction;
16) a process as in any one of points 1) to 15), in which step f) of isolating the palmitic acid triglyceride is conducted by means of preparative HPLC;
17) a process as in any one of points 1) to 16), in which the transesterification step g) of the palmitic acid triglyceride of step f) with a linear or branched C1-C4 alcohol, to give a C1-C4 alkyl ester of the palmitic acid and glycerol, is conducted in the presence of an acid catalyst, preferably selected from sulfuric acid, hydrochloric acid, an acid resin or a zeolite, using the C1-C4 alcohol as a solvent and heating to a temperature between 50° C. and 100° C., for example at the alcohol boiling temperature;
18) a process as in point 17), in which the palmitic acid triglyceride is transesterified with isopropanol to give isopropyl palmitate and glycerol in the presence of zeolites as a catalyst and at about 80° C. or reflux;
19) a process as in any one of points 1) to 18), in which step h) of reducing the glycerol obtained in step g) to give isopropanol is conducted in two steps: h1) reduction with hydrogen and a copper-ruthenium based catalyst supported on clay to give 1,2-propanediol and h2) reduction of 1,2-propanediol of step h1) with hydrogen and a copper-cerium based catalyst to give a mixture of propan-1-ol and propan-2-ol, followed by separation of propan-2-ol by distillation;
20) a process as in point 19), in which the isopropanol obtained in step h) is recycled in the transesterification step g) to obtain isopropyl palmitate.

These and any further objects, as outlined in the appended claims, will be described in the following description.

Further features and advantages of the invention will become apparent from the following description of preferred embodiments, given by way of non-limiting examples.

### BRIEF DESCRIPTION OF THE DRAWINGS

FIG. 1 shows a block diagram illustrating the process of the invention in a preferred embodiment.

### DETAILED DESCRIPTION OF THE INVENTION

The present invention is directed to a process for obtaining eicosapentaenoic acid (EPA) and docosahexaenoic acid (DHA) esters from algal material, comprising the steps of:
a) treating the algal material with an extraction solvent to give a liquid phase containing said solvent and algal oil and a solid phase comprising cellulose, sugars and proteins;
b) separating the solid phase from the liquid phase of step a);
c) separating the algal oil from the extraction solvent in the liquid phase of step b);
d) subjecting the algal oil of step c) to a low-temperature treatment so as to obtain the precipitation of saturated and monounsaturated fatty acid triglycerides and a liquid phase containing or consisting of polyunsaturated fatty acid triglycerides;
e1) separating, isolating and purifying DHA and EPA triglycerides from the liquid phase of step d), or
e2) transesterification with ethanol of DHA and EPA triglycerides and isolating the respective DHA and EPA ethyl esters;
f) optionally, isolating and purifying the palmitic acid triglyceride from the phase containing the saturated and monounsaturated fatty acid triglycerides of step d);
g) optionally, transesterification of the palmitic acid triglyceride of step f) with a linear or branched C1-C4 alcohol, to give a C1-C4 alkyl ester of the palmitic acid and glycerol;
h) optionally, hydrogenating the glycerol of step g) to give isopropanol.

The algal material used for the purposes of the present invention can be of various origin. Preferably, algal material from microalgae belonging to the genus Chlorella, Tetraselmis, Nannochloropsis and Scenedesmus will be used. The microalgae of the genus Nannochloropsis and Scenedesmus are preferred.

In the process of the invention a wet algal biomass can be used, obtained for example by separation from an algal suspension in water or aqueous solution (e.g., sea water), for example by microfiltration or centrifugation, or a dry algal biomass, obtained from the previous by means of a further drying step. The wet algal biomass which can be treated with DES in accordance with the process of the present invention preferably contains up to 90%, more preferably from over 5% up to 70%, by weight of water with respect to the total weight of the wet biomass. Conversely, the dry algal biomass can contain up to 5%, preferably up to 3% by weight of residual moisture.

The algal material in step a) is finely ground and contacted with the extraction solvent. The extraction solvent can be any solvent capable of extracting a bio-oil from the algal material. By way of example, a chloroform/methanol mixture can be used, for example in a 2:1 ratio.

Preferably, the extraction solvent is a solvent miscible in water.

In a preferred embodiment, the extraction solvent is a DES (Deep Eutectic Solvent). The term DES generally means a mixture of two or more components, at least one donor and one hydrogen bond acceptor, which interact with each other, for example by hydrogen bonds or even Van der Waals forces, associating to form a eutectic mixture having a melting temperature much lower than that of the components thereof. They are versatile, economical, environmentally compatible and biodegradable. The use of DES for the extraction of bio-oil from algae is known from WO2020/053118.

Low-melting temperature eutectics (Deep Eutectic Solvents (DESs) type III) are conveniently used, formed by an ammonium salt such as cholinium chloride, cholinium acetate, cholinium nitrate, betainium chloride, ethyl ammoniochloride, tetramethyl ammonium chloride, and a hydrogen-binding donor selected from a carboxylic acid such as acetic acid, formic acid, tartaric acid, oxalic acid, levulinic acid, citric acid or lactic acid, or an amine such as urea, thiourea, 1,3-dimethylurea, 1,1-dimethylurea, or a polyalcohol such as ethylene glycol, propylene glycol or glycerol. The ammonium salt and the hydrogen-binding donor are preferably in a molar ratio between 1:1 and 1:3.

In a particularly preferred embodiment the DES consists of a 1:3 ratio mixture of cholinium chloride and lactic acid or cholinium chloride and citric acid. The DES is obtained by mixing together the quaternary ammonium salt and the hydrogen-binding donor, in a molar ratio between 1:1 and 1:3 and heating the mixture thus obtained at a temperature of 80-100° C., for example for a period of about 30 minutes so as to obtain a clear and colorless liquid, which remains such once the DES is brought to a temperature close to room temperature.

In this preferred embodiment, step a) is conducted by treating the algal material with a weight amount of DES between 1:1 and 5:1, preferably between 1:1 and 2:1, with respect to the algal material, stirring the dispersion for a period between 30 minutes and 5 hours, preferably between 3 and 5 hours, at temperatures between 80° C. and 130° C., preferably between 90° C. and 110° C.

In step b), the separation of the solid phase can be conducted by filtration or centrifugation. The cellulose contained in the solid phase can be recovered and used for example as a precursor for industrial cellulose derivatives, such as nitro-cellulose or cellulose acetate, or as such for obtaining cellulose-based finished products, such as filters or paper materials.

In step c), the separation of the algal oil from the extraction solvent can be conducted by known methods, such as distillation or treatment with a strongly polar washing solvent, immiscible with the algal oil but miscible with the extraction solvent.

In a preferred embodiment, when the extraction solvent is a DES, the washing solvent is water. The washing solvent is added to the bio-oil solution in DES in a weight ratio preferably between 100 and 300% by weight, and the extraction is preferably conducted by stirring at room temperature. The supernatant organic phase consisting of bio-oil is then separated.

In certain embodiments, when the extraction solvent is a DES, step c) comprises a further step c1) of recycling the extraction solvent in step a). Such a step c1) comprises separating the extraction solvent from the washing solvent, for example by evaporation or distillation.

Step d), also known as “winterization”, consists of a fractional crystallization at low temperature for the separation of triglycerides of saturated and monounsaturated fatty acids (solid phase) from triglycerides of polyunsaturated fatty acids (liquid phase). The algal oil obtained in step c) is treated at a temperature between 0° C. and −30° C., preferably between −2° C. and −25° C., preferably in the presence of an alcohol and/or urea, more preferably an alcohol. The alcohol is preferably chosen from linear or branched C1-C4 alcohols, more preferably from ethanol, n-butanol and isopropanol. The weight ratio of alcohol with respect to the weight of the algal oil is preferably between 13:1 and 25:1.

Step e1) can preferably be conducted by means of preparative HPLC. For example, a reverse phase preparative HPLC system can be used, using either a gradient acetonitrile-chloroform mixture or a gradient methanol-water mixture as the mobile phase. In certain embodiments, a stationary phase consisting of a porous resin PMDVB (Poly-Meta-DiVinyl Benzene) modified with 3,5-dinitrobenzoylchloride modified with 2-chloro ethanol (W. K. Zhang et al. April 2001), or consisting of Silver(I)-mercaptopropyl, AgTCM (J. T. Dillon et al., J. Of Chromatography A 1312 August 2013) can be used. Preparative HPLC systems suitable for the present invention are for example described in Oh et al., Appl. Biol. Chem., (2020), 63:56, or in M. P. Mansour, J. Of Chromatography A, 1097 (2005) 54-58.

The alternative step e2) instead includes the transformation of the triglycerides of the PUFAs present in the liquid phase, in particular the DHA and EPA triglycerides, into the corresponding ethyl esters by transesterification. When step d) is conducted in the presence of ethanol, such a conversion can be advantageously conducted by adding to the liquid phase an acid catalyst such as sulfuric acid, hydrochloric acid, an acidic resin or a zeolite and heating at a temperature preferably between 70° C. and 90° C. until completion of the reaction. The reaction and gas chromatographic analytical determination can be conducted as for example described in Oh et al., Appl. Biol. Chem., (2020), 63:56.

The DHA and EPA ethyl esters can then be separated and purified by means of preparative HPLC as described above for step e1).

In certain embodiments, if required, PUFAs in the form of free fatty acid can be obtained by subjecting the obtained PUFA esters to a saponification reaction with alkaline or alkaline earth hydroxides, for example treatment with aqueous NaOH or KOH solutions. By neutralizing the sodium or potassium soap thus obtained, the corresponding fatty acid can be obtained in free form.

Step f) of isolating the palmitic acid triglyceride is preferably conducted by means of preparative HPLC, for example with one of the systems described for steps e1) and e2).

The step f) is carried out when the process of the present invention provides to produce the above esters of palmitic acid from algae simultaneously to the production of polyunsaturated fatty acids, in particular in the form of esters (simultaneous production).

The separation conditions and the choice of the stationary phase, the mobile phase and the gradient thereof in steps e1), e2) and f) can anyway be selected by those skilled in the art according to the conventional “trial-and-error” method.

The transesterification step g) of the palmitic acid triglyceride of step f) with a linear or branched C1-C4 alcohol, to give a C1-C4 alkyl ester of the palmitic acid and glycerol is conducted in the presence of an acid catalyst, using the C1-C4 alcohol as a solvent and heating to a temperature between 50° C. and 100° C., for example at the alcohol boiling temperature.

The acid catalyst can be sulfuric acid, hydrochloric acid, an acidic resin or a zeolite.

In a preferred embodiment, the palmitic acid triglyceride is transesterified with isopropanol to give isopropyl palmitate and glycerol. Such a reaction is preferably conducted in the presence of zeolites as a catalyst and at about 80° C. (isopropanol boiling temperature) or reflux.

The isopropyl palmitate obtained in step g) can be purified by distillation so as to obtain a cosmetic grade.

Step h) of reducing the glycerol obtained in step g) to give isopropanol is preferably conducted in two steps: h1) reduction with hydrogen and a copper-ruthenium based catalyst supported on clay to give 1,2-propanediol (method described in Green Chemistry 2009, 11, 1000-1006) and h2) reduction of 1,2-propanediol of step h1) with hydrogen and a copper-cerium based catalyst (method described in Green Chemistry 2016, 18, 782-791) to give a mixture of propan-1-ol and propan-2-ol, followed by separation of propan-2-ol by distillation.

The isopropanol obtained in step h) can be used directly in transesterification step g) if isopropyl palmitate is to be obtained.

It is also possible to use in the step g) only a portion or aliquot of the total amount of isopropanol obtained in step h), while using the remaining portion to 100 of the obtained isopropanol as additive for gasoline as known in the art of the gasoline additives.

In one embodiment of the process of the present invention, it is provided that the triglycerides of saturated and monounsaturated fatty acids obtained in form of precipitate in the step d) are fed to the step f), and to the subsequent optional steps g), h), only as a portion with respect to the total amount of the precipitate obtained in step d), for example in case that the market demand of isopropyl palmitate is very low while demanding high production of PUFA, thus entailing a decreased production of isopropyl palmitate only from the process of the present invention.

In that case, the remaining portion to 100 of the triglycerides of saturated and monounsaturated fatty acids (which is not submitted to step f)) can be optionally stored to be used subsequently or can be submitted as charge to another process downstream the present process such as, for example, a process for obtaining biodiesel by means of hydrodeoxygenation and hydroisomerisation of said charge.

An example of process to obtain biodiesel from renewable charge is the process of Ecofining as described in the patent applications WO2008113492, EP2134817A1, WO2015/181744, WO2020053118A1 herein incorporated integrally by reference.

In the Ecofining process, the charge containing the present mixture of triglycerides of saturated and monounsaturated fatty acids (i.e. the remaining portion with respect to the portion submitted to step f)) can undergo a first catalytic hydrodeoxygenation reaction, e.g. using, as catalyst, mixed sulphurs of cobalt and molibdenum, or of Ni—Mo, Ni—W, Co—W, supported on alumina: in this step the triglycerides are converted into alkanes and water whereas the glycerol present in the triglycerides is hydrogenated into propane.

Said catalytic hydrodeoxygenation reaction can be carried out by using hydrogen, at a pressure that ranges from 25 to 75 bar, preferably from 30 to 50 bar; the temperature of the catalytic hydrodeoxygenation reaction can ranges from 240° C. to 450° C., preferably from 270 to 430° C. Preferably in said reaction LHSV ranges from 0.5 to 2 h⁻¹, more preferably from 0.5 to 1 h⁻¹. The ratio H2/charge preferably ranges from 400 to 2000 N1/1.

Subsequently, in the second step of the Ecofining process, the alkanes obtained from the above hydrodeoxygenation reaction are hydroisomerised, in the presence of hydrogen, using an acid solid catalyst, thus obtaining branched alkanes which can be used as component of diesel fuel having a renewable origin.

An example of the acid solid catalyst usable in the hydroisomerization step is a catalytic system that includes a metallic component containing one or more metals of Group VIII, optionally in admixture with one or more metals of Group VIB, and an acid support (i.e. carrier) comprising a completely amorphous micro-mesoporous silica-alumina, preferably having a SiO₂/Al₂O₃ molar ratio ranging from 30 to 500, a surface area larger than 500 mg², a pore volume ranging from 0.3 to 1.3 ml/g, an average pore diameter smaller than 40 Angstrom, e.g. Pt/MSA, as described in EP2134817A1 herein integrally incorporated by reference.

The above hydroisomerization can be carried out at a temperature ranging from 250° C. to 450° C., preferably from 280 to 380° C.; the operating pressure can range from 25 to 70 bar, preferably from 30 to 50 bar. Preferably in said reaction LHSV ranges from 0.5 to 2 h⁻¹. The ratio H2/HC can preferably range from 200 to 1000 N1/1.

The amount of the proportion of the precipitate obtained in step d) which is not submitted to step f) is not binding of the scope of the present invention and it can vary within the range from 0,5% to 90%, preferably from 10% and 50%, with respect to the total amount of the precipitate obtained in step d), and it can also be up to 100% (i.e. no aliquot is submitted to step f)) without be thus outside the scope of the present invention.

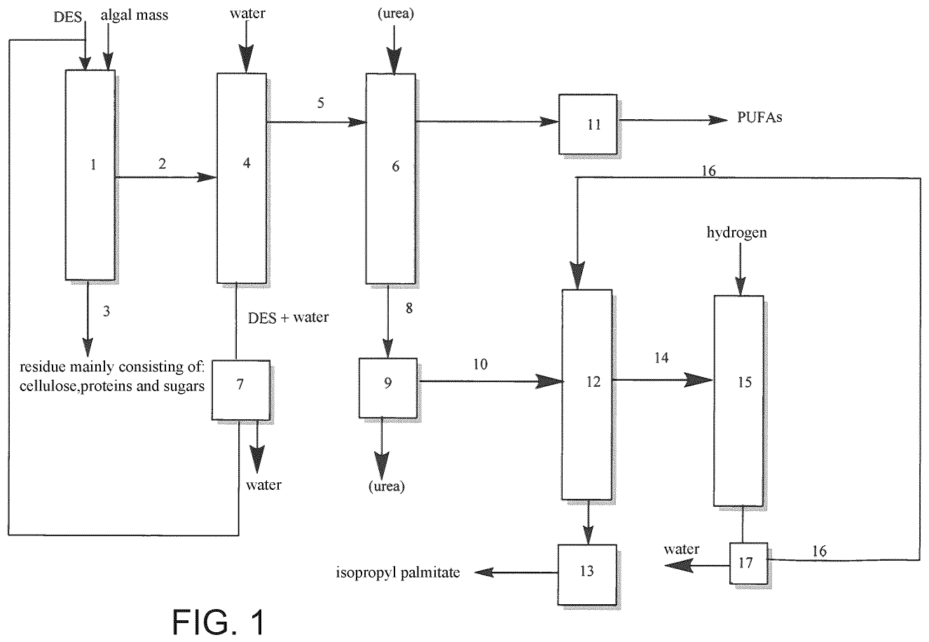

FIG. 1 shows a block diagram of the plant and process of the invention according to a preferred embodiment, in which:
1 indicates the mechanical pre-treatment (for example by means of a screw) for the extraction of bio-oil with DES;
2 indicates the phase consisting of bio-oil+DES and any water;
3 indicates the phase consisting of cellulose+sugars and proteins (extraction residue with DES);
4 indicates the DES/bio-oil hydrolysis and separation reactor with water;
5 indicates the phase consisting of bio-oil;
6 indicates the selective separation process between the saturated fatty acid phase and the polyunsaturated fatty acid phase (winterization);
7 indicates the evaporator of the DES+water mixture;
8 indicates the phase consisting of saturated fatty acid triglycerides;
9 indicates the purification of saturated fatty acids to obtain palmitic acid triglyceride (preparative HPLC);
10 indicates the phase consisting of the saturated fatty acid triglyceride;
11 indicates the separation and purification of PUFA triglycerides from the mixture of unsaturated fatty acid triglycerides;
12 denotes the transesterification reactor between saturated fatty acid triglycerides (e.g., palmitic acid) and an alcohol from the hydrogenation process from the reactor 15 (e.g., isopropanol);
13 indicates the separation and purification unit for the ester produced (e.g., isopropyl palmitate);
14 indicates the phase consisting of glycerol from the transesterification reactor;
15 indicates the catalytic hydrogenation reactor of glycerol to alcohol (isopropanol);
16 indicates the phase consisting of alcohol (isopropanol) from the hydrogenation reactor;
17 indicates the distillation and separation unit for the alcohol (isopropanol) from water and by-products.

### EXPERIMENTAL EXAMPLES

### Example 1 Extraction of Bio-Oil With Mechanical Grinding

4 g of Nannochloropsis CB-L2 are treated in a mechanical grinder together with 6 g of DES consisting of cholinium chloride and lactic acid (in weight ratio 1:3) at a temperature of 100° C., for a period of 210 minutes.

A fluid consisting of DES+bio-oil and a solid residue consisting of the oil-depleted algal mass is obtained.

The DES+bio-oil fluid is placed in a glass reactor together with 50 g of distilled water, stirred for 10 minutes and an oil phase supernatant is separated on the aqueous phase.

The oil phase is isolated and 840 g of algal oil are obtained (with a yield equal to 74% of the theoretical yield).

### Example 2 Extraction of Bio-Oil With Mechanical Grinding

4 g of Nannochloropsis CB-L2 are treated in a mechanical grinder together with 6 g of DES consisting of cholinium chloride and citric acid (in weight ratio 1:3) at a temperature of 100° C., for a period of 210 minutes.

A fluid consisting of DES+bio-oil and a solid residue consisting of the oil-depleted algal mass is obtained.

The DES+oil fluid is placed in a glass reactor together with 50 g of distilled water, stirred for 10 minutes and an oil phase supernatant is separated on the aqueous phase.

The oil phase is isolated and 744 g of algal oil obtained (with a yield equal to 74% of the are theoretical yield).

Example 3 Extraction of Bio-Oil With Mechanical Grinding 1 g of Scenedesmus SD-L1 are treated in a mechanical grinder together with 6 g of DES consisting of cholinium chloride and lactic acid (in weight ratio 1:3) at a temperature of 100° C., for a period of 210 minutes.

A fluid consisting of DES+bio-oil and a solid residue consisting of the oil-depleted algal mass is obtained.

The DES+oil fluid is placed in a glass reactor together with 50 g of distilled water, stirred for 10 minutes, an oil phase supernatant is separated on the aqueous phase.

The oil phase is isolated and 480 g of algal oil are obtained (with a yield equal to 100% of the theoretical yield).

### Example 4 Extraction of Bio-Oil With Mechanical Grinding

4 g of Scenedesmus SD-L1 are treated in a mechanical grinder together with 6 g of DES consisting of cholinium chloride and citric acid (in weight ratio 1:3) at a temperature of 100° C., for a period of 210 minutes.

A fluid consisting of DES+bio-oil and a solid residue consisting of the oil-depleted algal mass is obtained.

The DES+oil fluid is placed in a glass reactor together with 50 g of distilled water, stirred for 10 minutes and an oil phase supernatant is separated on the aqueous phase.

The oil phase is isolated and 345 g of algal oil are obtained (with a yield equal to 76% of the theoretical yield).

### Example 5 Separation of the PUFA-Rich Fraction

440 mg of algal bio-oil prepared as described in example 1 are placed in a 50 cc glass flask, then 6.8 g of anhydrous ethanol are added, followed by stirring to obtain a homogeneous solution.

The solution is cooled by placing it in a refrigerator at −5° C. and letting it stand overnight (about 12 h). The following morning, a solid precipitate is formed, which is cold filtered and consists of fraction the rich in saturated and monounsaturated fatty acids, which is then dried and a weight of 100 mg (21.9% of the total) is obtained. The clear solution is treated by evaporating the ethyl alcohol under vacuum, and a PUFA-rich fraction of the total weight of 334 mg (73.4%) is obtained. The relative compositions are determined by means of H-NMR.

### Example 6 Separation of the PUFA-Rich Fraction

463 mg of algal bio-oil prepared as described in example 1 are placed in a 50 cc glass flask, then 6.8 g of anhydrous ethanol are added, followed by stirring to obtain a homogeneous solution.

The solution is cooled by placing it in a refrigerator at −20° C. and letting it stand overnight (about 12 h). The following morning, a solid precipitate is formed, which is cold filtered and consists of the fraction rich in saturated and monounsaturated fatty acids, which is then dried and a weight of 133 mg (29.3% of the total) is obtained. The clear solution is treated by evaporating the ethyl alcohol under vacuum, and a PUFA-rich fraction of the total weight of 330 mg (72.8%) is obtained. The relative compositions are determined by means of H-NMR.

### Example 7 Separation of the PUFA-Rich Fraction

499 mg of algal bio-oil prepared as described in example 1 are placed in a 50 cc glass flask, then 7.5 g of anhydrous isopropanol are added, followed by stirring to obtain a homogeneous solution. The solution is cooled by placing it in a refrigerator at −5° C. and letting it stand overnight (about 12 h). The following morning, a solid precipitate is formed, which is cold filtered and consists of the fraction rich in saturated and monounsaturated fatty acids, which is then dried and a weight of 161 mg (32.2% of the total) is obtained. The clear solution is treated by evaporating the ethyl alcohol under vacuum, and a PUFA-rich fraction of the total weight of 345 mg (69.1%) is obtained. The relative compositions are determined by means of H-NMR.

### Example 8 Separation of the PUFA-Rich Fraction

374 mg of algal bio-oil prepared as described in example 1 are placed in a 50 cc glass flask, then 7.5 g of anhydrous n-butanol are added, followed by stirring to obtain a homogeneous solution.

The solution is cooled by placing it in a refrigerator at −5° C. and letting it stand overnight (about 12 h). The following morning, a solid precipitate is formed, which is cold filtered and consists of the fraction rich in saturated and monounsaturated fatty acids, which is then dried and a weight of 55 mg (15% of the total) is obtained. The clear solution is treated by evaporating the n-butyl alcohol under vacuum, and a PUFA-rich fraction of the total weight of 319 mg (85%) is obtained. The relative compositions are determined by means of H-NMR.

### Example 9 Synthesis of Isopropyl Palmitate

In a 250 ml bubble reflux flask, 25.6 g of palmitic acid (0.1 mol) are loaded, obtained by purifying the saturated fraction precipitated as described in example 5, to which 30.1 g of 2-propanol (0.5 mol) are added.

The 2-Propanol was obtained by catalytic hydrogenation of glycerin on copper-ruthenium supported on clay [as described in Green Chemistry 2009, 11, 1000-1006] to obtain 1,2-propanediol, followed by catalytic hydrogenation of 1,2-propanediol to a mixture of 1-propanol and 2-propanol with copper-cerium catalyst [as described in Green Chemistry 2016, 18, 782-791], from which 2-propanol is obtained by distillation separation.

To the mixture of palmitic acid and 2-propanol 8 g of Zerolit 225/H+ exchanger resin, dried at 100° C. and cooled, are added as an acid catalyst. The suspension is stirred and heated, refluxing it for four hours, then cooled and filtered to separate the exchanger resin.

The solution thus obtained is treated with a rotary evaporator to remove the unreacted 2 propanol, then the residue thus obtained is distilled by distilling the isopropyl palmitate (T.eb 162° C.).

25.37 g of pure isopropyl palmitate are obtained (yield 85% of the theoretical yield).

From the above, it is apparent that the process according to the invention achieves the pre-set objects. In fact, it is not only possible to isolate efficiently and with high purity PUFAs, in particular DHA and EPA in the form of esters, which can be used for the preparation of medicaments, supplements or foods for special medical purposes (FSMP) for cardio-protective goals, but it is also possible to simultaneously obtain a palmitic acid ester, in particular isopropyl palmitate, useful as an emollient or moisturizer in the cosmetic industry.

The possibility of using glycerol, obtained from the transesterification of palmitic acid triglycerides, for the reduction thereof to isopropanol also allows both an energy and raw material optimization and a reduction of waste material.

1. A process for obtaining eicosapentaenoic acid (EPA) and docosahexaenoic acid (DHA) esters from algal material, comprising the steps of:
a) treating the algal material with an extraction solvent to give a liquid phase containing said solvent and algal oil and a solid phase comprising cellulose, sugars and proteins;
b) separating the solid phase from the liquid phase of step a);
c) separating the algal oil from the extraction solvent in the liquid phase of step b);
d) subjecting the algal oil of step c) to a low-temperature treatment so as to obtain the precipitation of saturated and monounsaturated fatty acid triglycerides and a liquid phase containing or consisting of polyunsaturated fatty acid triglycerides;
e1) separating, isolating and purifying DHA and EPA triglycerides from the liquid phase of step d), or e2) transesterification with ethanol of DHA and EPA triglycerides and isolating the respective DHA and EPA ethyl esters;
f) optionally, isolating and purifying the palmitic acid triglyceride from the phase containing the saturated and monounsaturated fatty acid triglycerides of step d);
g) optionally, transesterification of the palmitic acid triglyceride of step f) with a linear or branched C1-C4 alcohol, to give a C1-C4 alkyl ester of the palmitic acid and glycerol;
h) optionally, hydrogenating the glycerol of step g) to give isopropanol.

2. The process according to claim 1, wherein the algal material is selected from algal material of microalgae belonging to the genus Chlorella, Tetraselmis, Nannochloropsis and Scenedesmus, the microalgae of the genus Nannochloropsis and Scenedesmus being preferred.

3. The process according to claim 1, wherein the extraction solvent is selected from a chloroform/methanol mixture, preferably in a 2:1 ratio, and a DES (Deep Eutectic Solvent), a DES being the preferred extraction solvent.

4. The process according to claim 3, wherein the extraction solvent is a low melting temperature eutectic (DES type III) formed by an ammonium salt selected from cholinium chloride, cholinium acetate, cholinium nitrate, betainium chloride, ethyl ammoniochloride, tetramethyl ammonium chloride, and a hydrogen-binding donor selected from a carboxylic acid such as acetic acid, formic acid, tartaric acid, oxalic acid, levulinic acid, citric acid or lactic acid, or an amine such as urea, thiourea, 1,3-dimethylurea, 1,1-dimethylurea, or a polyalcohol such as ethylene glycol, propylene glycol or glycerol, and wherein the ammonium salt and the hydrogen-binding donor are preferably in a molar ratio of 1:1 to 1:3.

5. The process according to claim 4, wherein DES consists of a 1:3 ratio mixture of cholinium chloride and lactic acid or cholinium chloride and citric acid.

6. The process according to claim 3, wherein step a) is conducted by treatment of the algal material with a weight amount of DES comprised between 1:1 and 5:1, preferably between 1:1 and 2:1, with respect to the algal material, stirring the dispersion for a period comprised between 30 minutes and 5 hours, preferably between 3 and 5 hours, at temperatures between 80° C. and 130° C., preferably between 90° C. and 110° C.

7. The process according to claim 1, wherein, in step b), the separation of the solid phase is conducted by filtration or centrifugation.

8. The process according to claim 1, wherein, in step c), the separation of the algal oil from the extraction solvent is conducted by distillation or by treatment with a strongly polar washing solvent, immiscible with the algal oil but miscible with the extraction solvent.

9. The process according to claim 8, wherein the extraction solvent is a DES and the washing solvent is water and wherein the washing solvent is added to the algal oil solution in DES in a weight ratio preferably between 100 and 300% by weight.

10. The process according to claim 9, wherein step c) comprises a further step cl) of recycling the extraction solvent in step a).

11. The process according to claim 1, wherein step d) consists of a “winterization” with low-temperature fractional crystallization of saturated and mono-unsaturated fatty acid triglycerides (solid phase) from the liquid phase containing poly-unsaturated fatty acid triglycerides, wherein the algal oil obtained in step c) is treated at a temperature between 0° C. and −30° C., preferably between −2° C. and −25° C., preferably in the presence of an alcohol and/or urea, more preferably an alcohol.

12. The process according to claim 11, wherein the alcohol is selected from linear or branched C1-C4 alcohols, and is preferably selected from ethanol, n-butanol and isopropanol, and wherein the weight ratio of alcohol with respect to the algal oil weight is preferably between 13:1 and 25:1.

13. The process according to claim 1, wherein step e1) is conducted by means of preparative HPLC.

14. The process according to claim 13, wherein step e1) is conducted by means of a reverse phase preparative system, using as mobile phase an acetonitrile-chloroform mixture with gradient, or a methanol-water mixture with gradient, and/or using a stationary phase consisting of a PMDVB porous resin (Poly-Meta-DiVinyl Benzene) modified with 3,5-dinitrobenzoyl chloride or modified with 2-chloro ethanol, or consisting of Silver(I)-mercaptopropyl (AgTCM).

15. The process according to claim 1, wherein the alternative step e2) comprises the transformation of the PUFA triglycerides present in the liquid phase, in particular DHA and EPA triglycerides, into the corresponding ethyl esters by transesterification, wherein, when step d) is conducted in the presence of ethanol, said conversion is conducted by adding to the liquid phase an acid catalyst, preferably selected from sulfuric acid, hydrochloric acid, an acid resin or a zeolite and heating to a temperature preferably between 70° C. and 90° C. until completion of the reaction.

16. The process according to claim 1, wherein step f) of isolating the palmitic acid triglyceride is conducted, preferably by means of preparative HPLC.

17. The process according to claim 1, wherein the transesterification step g) of the palmitic acid triglyceride of step f) with a linear or branched C1-C4 alcohol, to give a C1-C4 alkyl ester of the palmitic acid and glycerol, is conducted in the presence of an acid catalyst preferably selected from sulfuric acid, hydrochloric acid, an acid resin or a zeolite, using the C1-C4 alcohol as solvent and heating to a temperature comprised between 50° C. and 100° C., for example at the alcohol boiling temperature.

18. The process according to claim 17, wherein the palmitic acid triglyceride is transesterified with isopropanol to give isopropyl palmitate and glycerol in the presence of zeolites as a catalyst and at about 80° C. or reflux.

19. The process according to claim 1, wherein step h) of reducing the glycerol obtained in step g) to give isopropanol is conducted in two steps: h1) reduction with hydrogen and a copper-ruthenium based catalyst supported on clay to give 1,2-propanediol and h2) reduction of 1,2-propanediol of step h1) with hydrogen and a copper-cerium based catalyst to give a mixture of propan-1-ol and propan-2-ol, followed by separation of propan-2-ol by distillation.

20. The process according to claim 19, wherein the isopropanol obtained in step h) is recycled in the transesterification step g) to obtain isopropyl palmitate.

21. The process according to claim 16, wherein when step f) is carried out, only a portion of the total amount of saturated and monounsaturated fatty acid triglycerides obtained in form of precipitate in step d) is be submitted to step f) and subsequent optional steps g), h),
the remaining portion to 100 of said saturated and monounsaturated fatty acid triglycerides being optionally submitted, as charge, to a process obtaining a biodiesel by means of hyidrodeoxygenation and hydroisomerisation of said charge, preferably to the process of Ecofining.

22. An integrated process for the simultaneous production of eicosapentaenoic acid (EPA) and docosahexaenoic acid (DHA) esters, esters of the palmitic acid and biodiesel from an algal material, comprising
the process as defined in claim 1 wherein only a portion of saturated and monounsaturated fatty acid triglycerides obtained in step d) is submitted to step f); followed by subsequent
hydrodeoxygenation and hydroisomerization catalytic reactions of the remaining portion to 100 of said saturated and monounsaturated fatty acid triglycerides obtained in said step d).
